(19) United States
(12) Patent Application Publication
(10) Pub. No.: US 2015/0306030 A1
(43) Pub. Date: Oct. 29, 2015

(54) Method of Liquefying Solid Form of Medication

(71) Applicants: Theresa Benskin, Cumming, IA (US); Nina Aleksic, Broomfield, CO (US); Lucas Tigges, Colorado Springs, CO (US); Cody Metz, Cedar Rapids, IA (US)

(72) Inventors: Theresa Benskin, Cumming, IA (US); Nina Aleksic, Broomfield, CO (US); Lucas Tigges, Colorado Springs, CO (US); Cody Metz, Cedar Rapids, IA (US)

(21) Appl. No.: 14/695,684

(22) Filed: Apr. 24, 2015

Related U.S. Application Data
(60) Provisional application No. 61/983,743, filed on Apr. 24, 2014.

(51) Int. Cl. A61K 9/00

(52) U.S. Cl. CPC A61K 9/0095

(57) ABSTRACT

A method of liquefying solid form of medication into a liquefied medication solution is implemented by a medication liquefaction system and a removable medicine container. The solid form medication, which is difficult to swallow by itself, and a desired amount of drinkable fluid base product are added into the removable medicine container before the removable medicine container is hermetically attached with the medication liquefaction system. Then a carbon dioxide (CO₂) gas flow pressurizes the removable medicine container so that the solid form medication can be efficiently and quickly dissolve within the drinkable fluid base product in order to create the liquefied medication solution as the CO₂ gas flow carbonates the liquefied medication solution. Then the liquefied medication solution can be consumed by the user without any difficulties.

The current application claims a priority to the U.S. Provisional Patent application Ser. No. 61/983,743 filed on Apr. 24, 2014.

### FIELD OF THE INVENTION

The present invention relates generally to medical field. More specifically, the present invention is a method for liquefying oral solid forms of medication so that the liquefied medication can be easily swallowed by the respective patients.

### BACKGROUND OF THE INVENTION

The current clinical issue is addressed by the present invention is patient difficulty of swallowing oral solid medications, also called dysphagia. The present invention serves young children, senior citizens, institutionalized patients, and anyone that has difficulty swallowing medication. For example, a nationwide survey conducted by Harris Interactive reveals that 40% of American adults have experience difficulty swallowing pills. Of those who experience difficulty swallowing their medication, 14% have delayed taking doses of their medication, 8% have skipped a dose and 4% have discontinued using their medication. Therefore, about 50% of the prescriptions filled each year (3 billion) are taken incorrectly due to patients' inability to swallow pills which leads to about 9 million adverse drug reactions. This is also the result of 23% of all nursing home admission (380,000 patients) a cost of more than $31.3 billion each year. Non-compliance also causes 10% of all hospital admissions (3.5 million) and costs $15.2 billion annually. Current methods of making oral solid medications easier to administer usually involves the mechanical crushing of medicines. This method is disadvantageous for many reasons. First, it transforms medication into powder form which does not always provide an easier method for consumption. The process is also tedious and usually leaves large particles behind or loss of medicine powder, which causes a shortened dosage. As result, inefficient dosing can occur causing various complications for the patient. Also, most pill crushers are hard to use and can cause injury to the caregiver such as wrist sprain or palm bruising. Another method that has also been proposed for dissolving oral solid medications is the use of ultrasonic technology. These prototypes have been unsuccessful due to their relatively high price and their tendency to produce heat energy. Therefore, alternative solutions to these problems are much needed within the medical industry.

It is an objective of the present invention to provide a low-cost and simple medication liquefying method. Present herein is a medication liquefaction system that uses pressurized carbon dioxide (CO₂) power to liquefy pills for easier and efficient consumption. The user is likely be a nurse or caregiver who places a drinkable fluid base product and a medicine into a reusable or disposable bottle and then simply let the medication liquefaction system to transform the oral solid medication dosage and the drinkable fluid base product into a palatable and drinkable medication solution, thereby allowing the medication solution to be consumed by the patient. The present invention exceeds expectations compare to existing methods as it creates little to no heat energy, it is affordable, and it is portable. The present invention incorporates CO₂ pressure to break apart oral solid medication dosage and dissolves it into the palatable medication solution. Usage of the CO₂ is beneficial as it is relatively inexpensive, natural, abundant, colorless, odorless, non-flammable, and soluble. The present invention can be particularly suitable for use in a hospital setting, nursing home, or home used by a caregiver or a patient; however, it is not limited to such settings and can be utilized by anyone who has trouble swallowing pills. The present invention provides a solution to past device shortcoming by implementing a low-cost and simple method to liquefy medicines.

### BRIEF DESCRIPTION OF THE DRAWINGS

FIG. 1 is a basic illustration of the medication liquefaction system and the removable medicine container of the present invention.

FIG. 2 is a basic flowchart illustrating the overall process of the present invention.

FIG. 3 is a basic flowchart illustrating the volume ratio of the oral solid medication dosage and the desired amount of drinkable fluid base product within the overall process.

FIG. 4 is a basic flowchart illustrating the pressurization of the removable medicine container within the overall process.

FIG. 5 is a basic flowchart illustrating the first method of mixing the oral solid medication dosage and the desired amount of drinkable fluid base product within the overall process.

FIG. 6 is a basic flowchart illustrating the second method of mixing the oral solid medication dosage and the desired amount of drinkable fluid base product within the overall process.

### DETAIL DESCRIPTIONS OF THE INVENTION

All illustrations of the drawings are for the purpose of describing selected versions of the present invention and are not intended to limit the scope of the present invention.

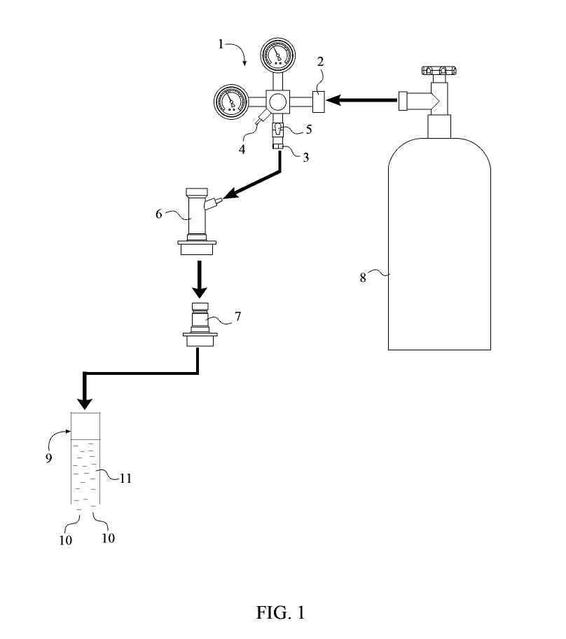

The present invention is a method of liquefying oral solid medication forms as the present invention is implemented by a medication liquefaction system and a removable medicine container 9. The medication liquefaction system is comprised of a pressure regulator 1, a disconnect coupler 6, a carbonated bottle cap 7, and a pressurized carbon dioxide (CO₂) tank 8. In reference to FIG. 1, the general configuration of the medication liquefaction system, the pressure regulator 1 is in fluid communication with the pressurized CO₂ tank 8 while the carbonated bottle cap 7 is in fluid communication with the pressure regulator 1 through the disconnect coupler 6. A housing may enclose the pressure regulator 1, the pressurized CO₂ tank 8, and the disconnect coupler 6 in order to provide an aesthetically pleasing unit; however, the carbonated bottle cap 7 traverses through the housing so that the removable medicine container 9 can be externally attached with the carbonated bottle cap 7 to liquefy oral solid medication forms. An automated system of the medication liquefaction system may comprise a control module, a power source, and an external switch or a control panel as the control module allows the user to operate the pressurized CO₂ tank 8 and the pressure regulator 1 through the external switch or the control panel. The power source is electrically connected with the external switch and the control module so that the medication liquefaction system can automatically liquefy oral solid medication forms into a drinkable slurry or solution according to the system parameters. A manual system of the medication liquefaction system generally excludes the control module, the power source, and the external switch or the control panel as the pressurized CO₂ tank 8 and the pressure regulator 1 are physically operated by the user.

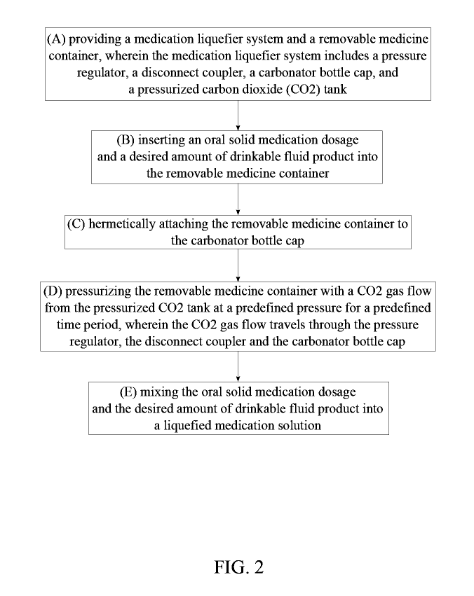

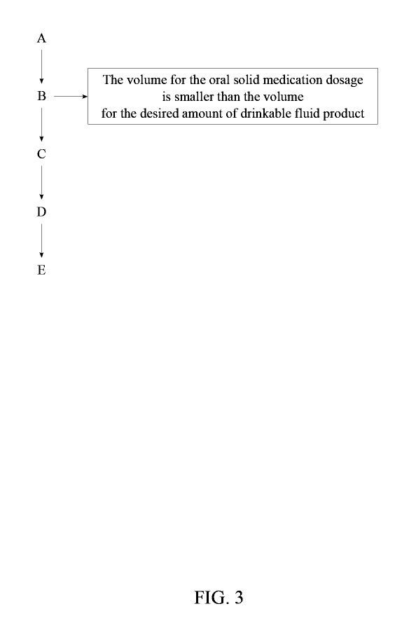

In order to start the present invention, the user first needs to insert an oral solid medication dosage 10 and a desired amount of drinkable fluid base product 11 into the removable medicine container 9 as shown in FIG. 2. The oral solid medication dosage 10 within the present invention represent oral solid medication forms that the user may have a difficulty swallowing. During the insertion of the oral solid medication dosage 10, the user can break the oral solid medication dosage 10 into smaller pieces as the smaller pieces may speed up the overall process of the present invention. The desired amount of drinkable fluid base product 11 utilized within the present invention can include, but is not limited to, water, sugar water, fruit juice, flavored water, and any other type liquid that minimize any unwanted taste of the oral solid medication dosage 10. In reference to FIG. 2 and FIG. 3, the present invention preferably requires a smaller volume of the oral solid medication dosage 10 compare to the volume of the desired amount of drinkable fluid base product 11 in order to maximize the efficiency of the present invention. Once the correct ratio of the oral solid medication dosage 10 and the desired amount drinkable fluid base product 11 are added together, the initial dissolving phase begins within the removable medicine container 9 as the oral solid medication dosage 10 slowly begins to dissolve in the desired amount of drinkable fluid base product 11.

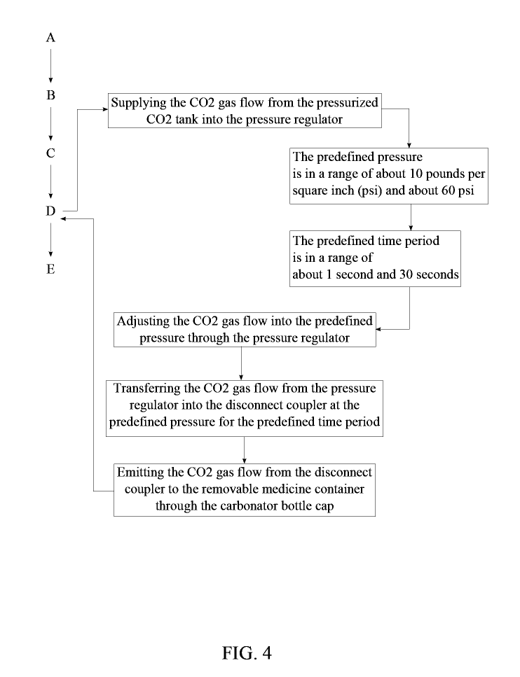

The removable medicine container 9 is then hermetically attached to the carbonator bottle cap 7 of the medication liquefaction system as shown in FIG. 2. The airtight seal between the removable medicine container 9 and the carbonator bottle cap 7 is critical to the present invention so that the efficiency can be maximized within the medication liquefaction system. In reference to FIG. 2 and FIG. 4, once the removable medicine container 9 is secured to the medication liquefaction system, the removable medicine container 9 is pressurized within with a CO₂ gas flow from the pressurized CO₂ tank 8 at a predefined pressure for a predefined time period. More specifically, the CO₂ gas flow is supplied from the pressurized CO₂ tank 8 into the pressure regulator 1 through an inlet connection 2 of the pressure regulator 1. The pressure regulator 1 is then able to adjust the CO₂ gas flow into the predefined pressure and then transfers the CO₂ gas flow into the disconnect coupler 6 for the predefined time period, where the CO₂ gas flow stays constant with the predefined pressure. An outlet connection 3 of the pressure regulator 1 is utilized to provide the in fluid connection between the disconnect coupler 6 and the pressure regulator 1. Then the CO₂ gas flow from the disconnect coupler 6 is emitted into the removable medicine container 9 through the carbonated bottle cap 7 as the removable medicine container 9 is able to withstand the high pressure fluid flow of the present invention. Additionally, the usage of the CO₂ is beneficial to the present invention as it is relatively inexpensive, natural, abundant, colorless, odorless, non-flammable, and soluble in the desired amount of drinkable fluid base product 11.

In reference to FIG. 4, the predefined pressure of the present invention ranges from 10 pounds per square inch (psi) to 60 psi, preferably about 15 psi and 55 psi, more preferably about 30 psi and about 50 psi. However, the most preferred predefined pressure of the present invention is about 45 psi. The predefined time period of the present invention ranges from 1 second to 30 seconds, preferably about 1 second and 20 seconds, more preferably about 1 second and 15 seconds, where the most preferred predefined time period is about 10 second or less. The medication liquefaction system may generally employ particular temperature parameter from 15 degree Celsius (° C.) to 45° C., preferably about 20° C. and 40° C., more preferably about 30° C. and 37° C., where the most preferred particular temperature parameter is about 35° C.

Preferably, the medication liquefaction system respectively displays an inlet pressure of the pressure regulator 1, an outlet pressure of the pressure regulator 1, the predefined time period, particular temperature parameters through individual gages or a display panel so that the user can be visually informed about the operating parameters of the present invention.

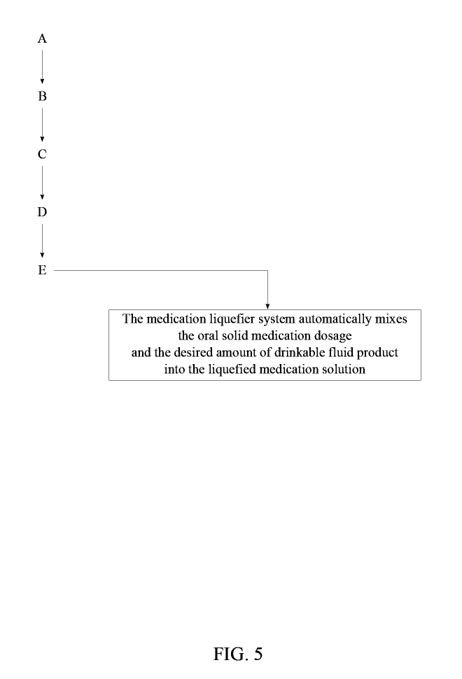

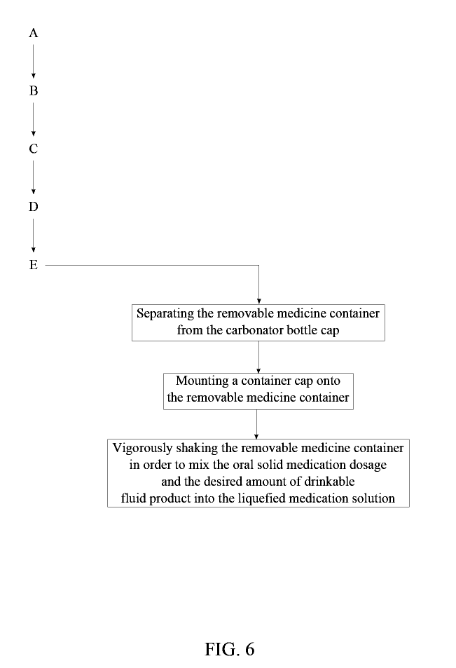

In reference to FIG. 2, once the CO₂ gas flow is discharged into the removable medicine container 9, the oral solid medication dosage 10 and the desired amount of drinkable fluid base product 11 are mixed together in order to form a liquefied medication solution. The discharge of the CO₂ gas flow into the removable medicine container 9 creates swirling of the desired amount of drinkable fluid base product 11 as the desired amount of drinkable fluid base product 11 flows around the oral solid medication dosage 10. Simultaneously, the CO₂ gas flow is also dissolved into the liquefied medication solution, carbonating the liquefied medication solution. The mixing process of the present invention can be carried out with two different methods, where one does not precede the other. In reference to FIG. 5, the first mixing process of the present invention is implemented through the medication liquefaction system; More specifically, the medication liquefaction system automatically mixes the oral solid medication dosage 10 into the desired amount of drinkable fluid base product 11 by shaking the removable medicine container 9 so that the liquefied medication solution can be obtained through the present invention. In reference to FIG. 6, the second mixing process of the present invention is implemented by the user, where the user first separates the removable medicine container 9 from the carbonator bottle cap 7. Then a container cap is mounted onto the removable medicine container 9 so that the dissolving oral solid medication dosage 10 and the desired amount of drinkable fluid base product 11 can be trapped within the removable medicine container 9. Then the removable medicine container 9 is vigorously shaken in order to form the liquefied medication solution as the oral solid medication dosage 10 completely dissolves into the desired amount of drinkable fluid base product 11.

In reference to FIG. 1, the medication liquefaction system further comprises a shut-off valve 5 and a relief valve 4, where the shut-off valve 5 and the relief valve 4 are in fluid communication with the pressure regulator 1. More specifically, the shut-off valve 5 traverses through the housing and is externally positioned with the housing so that the user is able to operate the shut-off valve 5 when necessary. The shut-off valve 5 functions as an emergency switch within the medication liquefaction system so that the CO₂ gas flow can be stopped before the removable medicine container 9. The relief valve 4 functions as a safety valve within the medication liquefaction system. Since over pressurization of the removable medicine container 9 can cause fracture or explosion, the relief valve 4 automatically becomes operational if the outlet pressure or the inlet pressure of pressure regulator 1 exceeds a certain threshold of the relief valve 4.

The present invention creates little to no heat energy within the medication liquefaction system and creates a portable system that can be easily operated. The medication liquefaction system is also relatively a noise free apparatus that can use in a hospital setting, nursing home, or home.

Although the invention has been explained in relation to its preferred embodiment, it is to be understood that many other possible modifications and variations can be made without departing from the spirit and scope of the invention as hereinafter claimed.

What is claimed is:

1. A method of liquefying solid form of medication comprises:
(A) providing a medication liquefaction system and a removable medicine container, wherein the medication liquefaction system includes a pressure regulator, a disconnect coupler, a carbonator bottle cap, and a pressurized carbon dioxide (CO₂) tank;
(B) inserting a oral solid medication dosage and a desired amount of drinkable fluid base product into the removable medicine container;
(C) hermetically attaching the removable medicine container to the carbonator bottle cap;
(D) pressurizing the removable medicine container with a CO₂ gas flow from the pressurized CO₂ tank at a predefined pressure for a predefined time period, wherein the CO₂ gas flow travels through the pressure regulator, the disconnect coupler and the carbonator bottle cap; and
(E) mixing the oral solid medication dosage and the desired amount of drinkable fluid base product into a liquefied medication solution.

2. The method of liquefying solid form of medication as claimed in claim 1, wherein the volume for the oral solid medication dosage is smaller than the volume for the desired amount of drinkable fluid base product.

3. The method of liquefying solid form of medication as claimed in claim 1, wherein the predefined pressure is in a range of about 10 pounds per square inch (psi) and about 60 psi.

4. The method of liquefying solid form of medication as claimed in claim 1, wherein the predefined time period is in a range of about 1 second and 30 seconds.

5. The method of liquefying solid form of medication as claimed in claim 1 comprises:
supplying the CO₂ gas flow from the pressurized CO₂ tank into the pressure regulator;
adjusting the CO₂ gas flow into the predefined pressure through the pressure regulator;
transferring the CO₂ gas flow from the pressure regulator into the disconnect coupler at the predefined pressure for the predefined time period; and
emitting the CO₂ gas flow from the disconnect coupler to the removable medicine container through the carbonator bottle cap.

6. The method of liquefying solid form of medication as claimed in claim 1, wherein the medication liquefaction system automatically mixes the oral solid medication dosage and the desired amount of drinkable fluid base product into the liquefied medication solution.

7. The method of liquefying solid form of medication as claimed in claim 1 comprises:
separating the removable medicine container from the carbonator bottle cap;
mounting a container cap onto the removable medicine container; and
vigorously shaking the removable medicine container in order to mix the oral solid medication dosage and the desired amount of drinkable fluid base product into the liquefied medication solution.
